(19) United States
(12) Patent Application Publication
(10) Pub. No.: US 2005/0055096 A1
(43) Pub. Date: Mar. 10, 2005

(54) Functional spinal unit prosthetic

(75) Inventors: Hassan A. Serhan, S.Easton, MA (US); Robert Labrom, Brisbane (AU); John R. Hawkins, Cumberland, RI (US); William Dunbar, Norton, MA (US); Amie Borgstrom, North Attleborough, MA (US); Seungkyu Daniel Kwak, Grafton, MA (US); Andrew Dooris, Fall River, MA (US); Michael A. Slivka, Taunton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: DePuy Spine, Inc., Raynham, MA (US)

(21) Appl. No.: 10/850,280

(22) Filed: May 20, 2004

Related U.S. Application Data
(63) Continuation-in-part of application No. 10/334,601, filed on Dec. 31, 2002.

(51) Int. Cl. A61F 2/44

(52) U.S. Cl. 623/17.11; 606/61

(57) ABSTRACT

A facet joint replacement system having an elongate rod and a polyaxial screw having a groove therein, wherein the rod is slidably received within the groove of the polyaxial screw.

### RELATED DATA

This application claims the benefit of U.S. Ser. No. 10/334,601, filed Dec. 31, 2002, and entitled” Prosthetic Facet Joint Ligament (Attorney Docket DEP5014), the specification of which is incorporated by reference in its entirety.

### BACKGROUND OF THE INVENTION

One of the most common surgical interventions today is arthrodesis, or spine fusion, in which two or more adjacent vertebral bodies are fused together in order to alleviate pain associated with the disc(s) located between those vertebral bodies. Approximately 300,000 such procedures are performed annually in the United States alone. Clinical success varies considerably, depending upon technique and indications, and consideration must be given to the concomitant risks and complications.

While spine fusion generally helps to eliminate certain types of pain, it has also been shown to decrease function by limiting the range of motion for patients in flexion, extension, rotation and lateral bending. Furthermore, it is believed that spine fusion creates increased stresses on (and, therefore, accelerated degeneration of) adjacent non-fused motion segments. Additionally, pseudoarthrosis, resulting from an incomplete or ineffective fusion, may reduce or even totally eliminate the desired pain relief for the patient. Also, the fusion device(s) used to effect fusion, whether artificial or biological, may migrate out of the fusion site, thereby creating significant new problems for the patient. Lastly, the recuperation time after a fusion procedure can be lengthy.

Recently, several attempts have been made to recreate the natural biomechanics of the spine through the use of an artificial disc. Artificial discs are intended to restore articulation between vertebral bodies so as to recreate the full range of motion normally allowed by the elastic properties of the natural disc, which directly connects two opposed vertebral bodies. However, the artificial discs developed to date do not fully address the mechanics of motion of the spinal column.

In addition to the foregoing, posterior elements called the facet (or zygapophyseal) joints help to support axial, torsional and shear loads that act on the spinal column. Furthermore, the facet joints are diarthroidal joints that provide both sliding articulation and load transmission features. The facet's articular surfaces contact in extension, limiting rotation and increasing compressive load. The articular surfaces also contact on one side of the spine in lateral bending and axial rotation, also limiting rotation and transferring load.

However, the facet joints can also be a significant source of spinal disorders and, in many cases, debilitating pain. The articular cartilaginous surfaces can degenerate due to mechanical or biological factors and cause pain as with other joint osteoarthritis, or enlarge and produce stenosis. For example, a patient may suffer from arthritic facet joints, severe facet joint tropism or otherwise deformed facet joints, facet joint injuries, etc. There is currently a lack of suitable intervention procedures for facet joint disorders. Facetectomy, or the removal of the facet joints, may provide some relief, but is also believed to significantly decrease the stiffness of the spinal column (i.e., hypermobility) in all planes of motion: flexion and extension, lateral bending, and rotation. Furthermore, problems with the facet joints can also complicate treatments associated with other portions of the spine. By way of example, contraindications for artificial discs include arthritic facet joints, absent facet joints, severe facet joint tropism or otherwise deformed facet joints. Accordingly, there is a need for a facet joint replacement that addresses these concerns.

U.S. Pat. No. Re. 36,758 (Fitz I) discloses an artificial facet joint where the inferior facet, the mating superior facet, or both, are simply covered with a cap. Because placement of the cap requires no preparation of the bone or articular surfaces; it covers and, therefore, preserves the bony and articular structures.

However, simple capping of the facet has several potential disadvantages. If the facet joint is osteoarthritic, a cap will not remove the source of the pain. Additionally, at least in the case of surface replacements for osteoarthritic femoral heads, the capping of articular bone ends has proven to lead to clinical failure due to mechanical loosening. This clinical failure is hypothesized to be a consequence of disrupting the periosteum and ligamentum teres femoris, both of which play a role in delivering nutrition to the femoral head, thereby leading to avascular necrosis of the bony support structure for the surface replacement. It is possible that corresponding problems could develop from capping the facet. Another potential disadvantage of facet capping is that in order to accommodate the wide variability in anatomical morphology of the facets, not only between individuals but also between levels within the spinal column, as well as due to associated hypertrophic and degenerative changes, a very wide range of cap sizes and shapes is required, or significant reshaping.

U.S. Pat. No. 6,132,464 (“Martin”) describes a replacement of the articular surfaces and means for supporting and fixing these replacements to the posterior processes. The articulating surface itself is described as having “the shape, position, and orientation of a natural articular facet”. It discloses a spinal facet joint prosthesis that is supported on the lamina (which is sometimes also referred to as the posterior arch). Extending from this support structure are inferior and/or superior blades that replace the cartilage at the facet joint. The prosthesis of U.S. Pat. No. 6,132,464 generally preserves existing bony structures and therefore does not address pathologies which affect the bone of the facets in addition to affecting the associated cartilage. Furthermore, the prosthesis of U.S. Pat. No. 6,132,464 requires a secure mating between the prosthesis and the lamina. However, the lamina is a very complex and highly variable anatomical surface. As a result, in practice, it is very difficult to design a prosthesis that provides reproducible positioning against the lamina so as to correctly locate the cartilage-replacing blades for the facet joints.

The U.S. Pat. No. 6,132,464 patent describes articular surfaces and means of attachment, but does not describe a capsular replacement.

U.S. Pat. No. 5,571,191 (“Fitz II”) describes a facet prosthesis comprising superior and inferior components, pyramidal or conical in shape, fitting over the facet processes, and having low friction mating surfaces. Although this patent describes articular surfaces and means of attachment, it does not describe a capsular replacement.

Gardner et al. Eur. Spine J (2002) (Supp 2): S157-163, discloses Graf ligamentoplasty as a means of stabilizing and reducing mobility of one or more severely symptomatic motion segments associated with degenerative disc disease. FIG. 1 shows Polyester bands wrapped around a pair of pedicle screws extending from adjacent vertebral bodies. This ligament also appears to be disclosed in U.S. Pat. No. 5,092,866 (“Breard”). According to Gardner, appropriate Graf bands immobilizes the motion segment in lordosis with the facet joints in a position of full extension, in which position they are very stable. See page S159. Accordingly, Graf ligamentoplasty essentially immobilizes the facet joint. Gardner does not disclose a mobile ligament that traverses a facet joint.

Senegas et al., Eur. Spine J. (2002) 11 (Supp 2): S164-9 discloses a Wallis implant system comprising a titanium interspinous blocker and a Dacron ligament, wherein the blocker is placed between two spinous processes and the Dacron ligament wraps around spinous processes. See p. S165. Accordingly, Senegas does not disclose a ligament that traverses a facet joint.

WIPO PCT Published Patent Application No. WO 00/53126 (“Ogun”) discloses a memory metal implant for fixing an articulated joint, including a facet joint.

The Dynesys system is generally used as a replacement for the natural posterior longitudinal ligament. The system includes a cable housed inside a plastic sheath, and is attached to superior and inferior pedicles. The ligament of the Dynesys system does not traverse a facet joint.

US Published Patent Application No. 2003/0004572 (“Goble”) discloses a prosthesis comprising an intervertebral disc prosthesis and a fact joint prosthesis. Goble does not disclose a facet joint ligament. FIG. 12 of Goble discloses a facet joint replacement system wherein each of the superior and inferior components are fixed respectively to the upper and lower pedicles. However, the superior component of the Goble system has no adjustability (i.e., the screw-articulation surface distance is fixed). Second, the articulation surface appears to be set higher than the pedicle screw securing the inferior component. In such a case, long term bearing may cause twirling of the inferior component about the lower pedicle screw axis.

### SUMMARY OF THE INVENTION

The present inventors have appreciated that natural facet joints are true articulating joints in which the facet joint capsule and surrounding ligaments play a very important role. While the articular surface of the joint transfers compression, the facet joint capsule transfers tension. In flexion, the joint opens and the facet joint capsule and the supraspinous ligament (SSL) is stretched. Several biomechanical in vitro studies have demonstrated the contribution of the capsule and surrounding ligaments to total motion segment stiffness in flexion. Replacing the articular surface may relieve pain, but does not fully restore joint functionality. Accordingly, the present inventors recognized a need for stabilizing the facet joint in tension.

In one aspect of the present invention, the patient's natural intervertebral disc is replaced with a prosthetic motion disc, and the patient's natural facet joint is replaced with superior and inferior bearing components that are then stabilized in tension by a prosthetic ligament having fasteners fixated either in the superior and inferior vertebrae or in superior and inferior prosthetic facet joint components.

Accordingly, the present invention, in a first part, replaces the natural but diseased disc with an artificial motion disc that more fully provides the natural mechanical relationship provided by a natural healthy intervertebral disc. Accordingly, the disc component of the present invention more closely simulates physiological contributions of the intervertebral disc and so more closely approximates a full natural disc.

Also, the present invention, in a second part, replaces the natural facet joint capsule with an artificial construct that more fully provides the natural mechanical relationship provided by a natural healthy facet joint. In particular, by providing a ligament that stretches while resisting tension, thus increasing joint stability, the present invention more closely simulates physiological contributions of the facet joint capsule and so more closely approximates a full natural facet joint.

Therefore, in accordance with the present invention, there is provided a kit for providing therapy to a functional spinal unit, the FSU comprising an upper vertebra Vu having an upper vertebral body VB_{U} and an upper facet F_{U}, a lower vertebra having a lower vertebral body VB_{L} and a lower facet F_{L}, the vertebral bodies defining a disc space therebetween, the upper and lower facets defining a facet joint FJ, the kit comprising:
a) a motion disc adapted for insertion into the disc space,
b) a facet joint replacement (“FJR”) adapted to replace at least a portion of a natural facet joint comprising first and second facets, and
c) a ligament adapted to constrain relative movement between the facets.

### DESCRIPTION OF THE FIGURES

FIG. 1 is a side view of the present invention implanted in a human spine.

FIG. 2 is a posterior view of the present invention implanted in a human spine.

FIG. 3 is a 3-piece motion disc component of the present invention.

FIG. 4 is a 2-piece motion disc component of the present invention.

FIG. 5 is a generic articulating facet joint replacement component of the present invention.

FIG. 6 is a preferred articulating facet joint replacement component of the present invention.

FIG. 7 is cushion-type facet joint replacement component of the present invention including.

FIGS. 8a, 8b and 8d are generic ligament components of the present invention.

FIG. 8c is a fastener component of the generic ligament component of the present invention.

FIG. 9 is a preferred ligament component of the present invention.

FIGS. 10a and 10b are depictions of a functional spinal unit (FSU) of a human spine.

FIGS. 11a-11e are posterior views of a minimally invasive facet joint replacement system.

FIG. 12 is a side view of a ball-and-socket type facet joint replacement system.

### DETAILED DESCRIPTION OF THE INVENTION

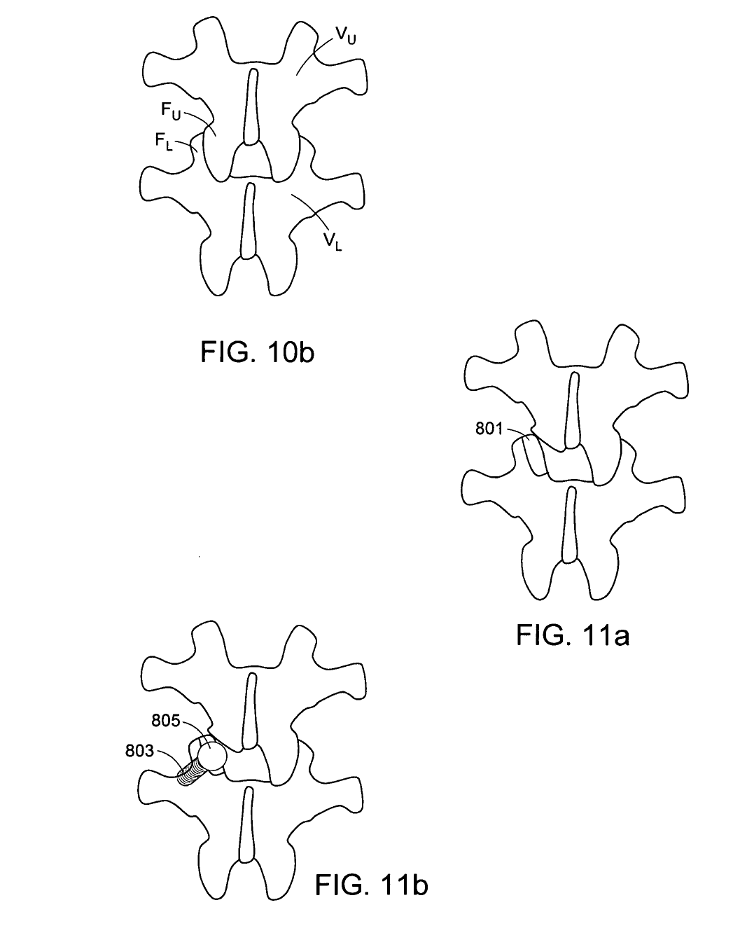

Now referring to FIGS. 10a and 10b, there is provided an anatomic “functional spinal unit” or FSU comprising an upper vertebrae having an upper vertebral body Vu and an upper facet Fu, a lower vertebra having a lower vertebral body V_{L} having a lower facet F_{L}. The vertebral bodies lies in the anterior A portion of the FSU, while the facets lie in the posterior portion P of the FSU. Disposed between the vertebral bodies is a disc space DISC. Disposed between the facets is an “facet joint”. The supraspinous ligament SSL lies posterior to the spinous processes. The Posterior longitudinal ligament PLL lies posterior to the vertebral bodies.

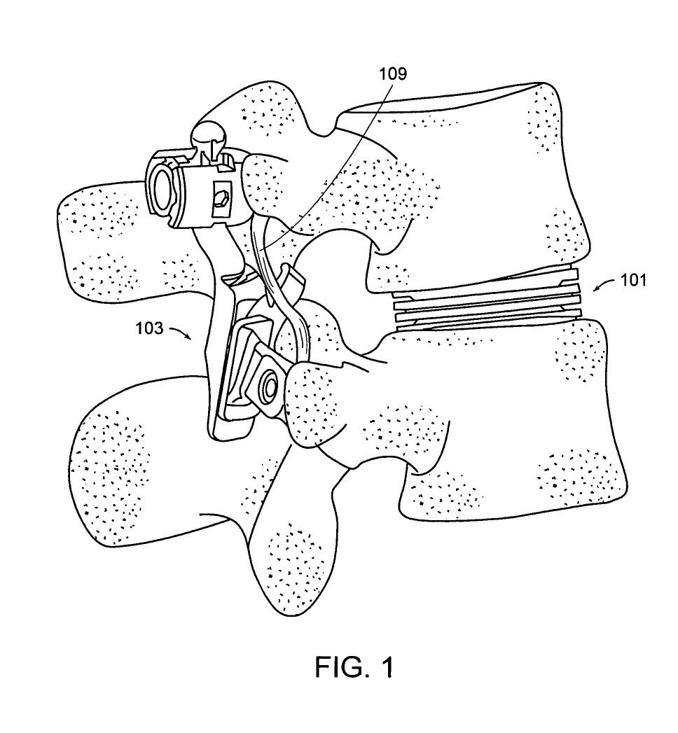

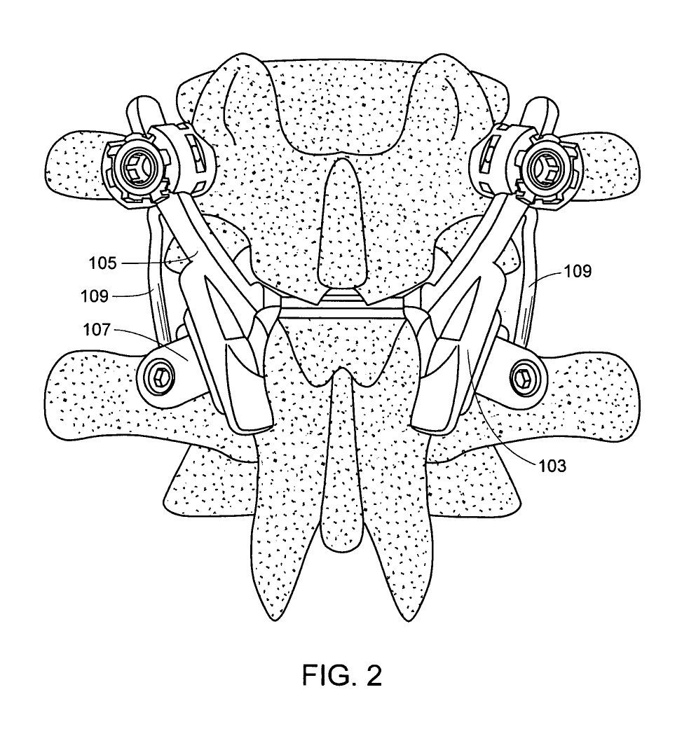

Now referring to FIGS. 1 and 2, there is provided a a kit for providing therapy to a functional spinal unit, the FSU comprising an upper vertebra having an upper vertebral body V_{U} and an upper facet F_{U}, a lower vertebra having a lower vertebral body VB_{L} and a lower facet F_{L}, the vertebral bodies defining a disc space therebetween, the upper and lower facets defining a facet joint FJ, the kit comprising:
a) a motion disc 101 adapted for insertion into the disc space,
b) a facet joint replacement system 103 comprising superior 105 and inferior 107 components, the system adapted to replace at least a portion of a natural facet joint comprising first and second facets, and
c) a ligament 109 adapted to constrain relative movement between the facets.

The motion disc component of the present invention can be any prosthetic capable of at least partially restoring the natural motions of the intervertebral disc. In preferred embodiments, the motion disc is selected from the group consisting of an articulating disc, a cushion disc and a spring-based disc. Various motion discs are described by Stefee et al. in U.S. Pat. No. 5,071,437; Gill et al. in U.S. Pat. No. 6,113,637; Bryan et al. in U.S. Pat. No. 6,001,130; Hedman et al. in U.S. Pat. No. 4,759,769; Ray in U.S. Pat. No. 5,527,312; Ray et al. in U.S. Pat. No. 5,824,093; Buttner-Janz in U.S. Pat. No. 5,401,269; and Serhan et al. in U.S. Pat. No. 5,824,094; all which documents are hereby incorporated herein by reference in their entireties.

Preferred articulating motion devices are disclosed in U.S. Pat. Nos. 5,556,431 and 5,674,296, the specifications of which are incorporated by reference.

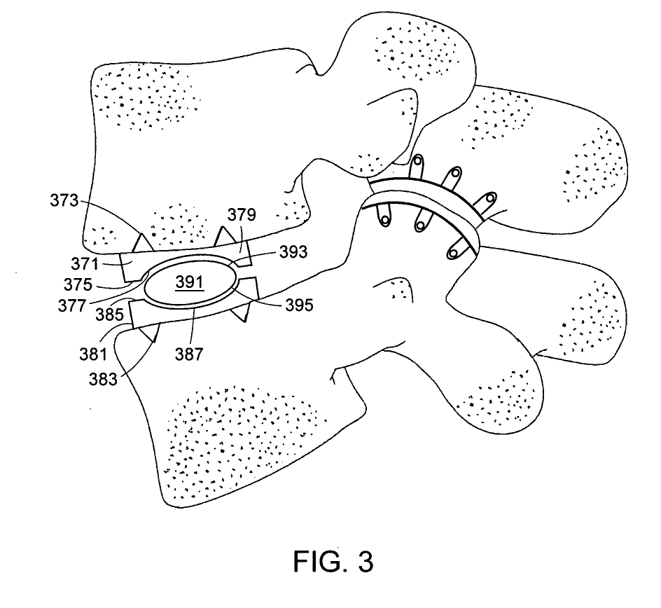

In some embodiments, the articulating motion disc is a three-piece design comprising two endplates and a core. Now referring to FIG. 3, in some embodiments, the articulating three-piece motion disc comprises:
a) a first prosthetic vertebral endplate 371 comprising:
   i) an outer surface 373 adapted to mate with a first vertebral body,
   ii) an inner surface 375 having a first articulation surface 377,
   iii) a body portion 379 connecting the inner and outer surfaces,
b) a second prosthetic vertebral endplate 381 comprising:
   i) an outer surface 383 adapted to mate with a second vertebral body, and
   ii) an inner surface 385 comprising a first articulation surface 387,
c) a core member 391 comprising:
   i) a first articulation surface 393 adapted for articulation with the first articulation surface of the first endplate, and
   ii) a second articulation surface 395 adapted for articulation with the first articulation surface of the second endplate,
      wherein the core member is oriented to produce a first articulation interface between the first articulation surface of the first endplate and the first articulation surface of the core member, and a second articulation interface between the first articulation surface of the second endplate and the second articulation surface of the core member.

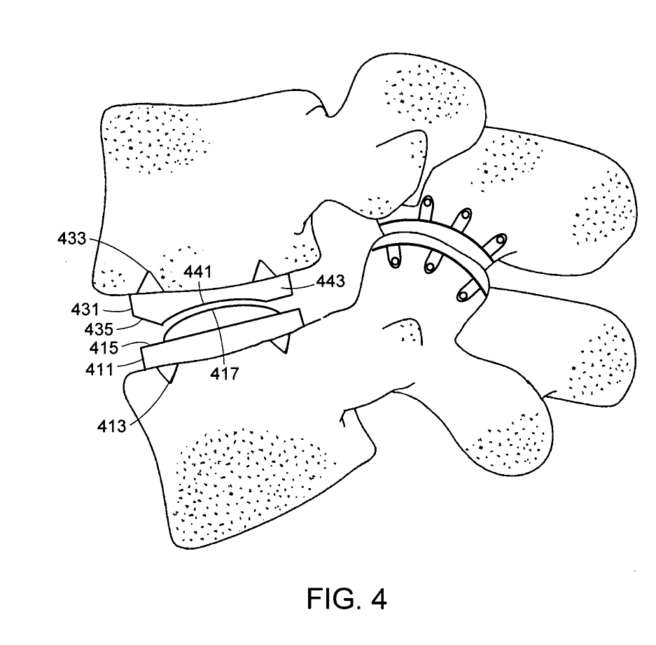

In some embodiments, the articulating motion disc is a two-piece design comprising two endplates. Now referring to FIG. 4, in some embodiments, the articulating two-piece motion disc 401 comprises:
a) a first prosthetic vertebral endplate 431 comprising:
   i) an outer surface 433 adapted to mate with a first vertebral body,
   ii) an inner surface 435 having a first articulation surface 441,
   iii) a body portion 443 connecting the inner and outer surfaces,
b) a second prosthetic vertebral endplate 411 comprising:
   i) an outer surface 413 adapted to mate with a second vertebral body, and
   ii) an inner surface 415 comprising a second articulation surface 417,
      wherein the first and second articulation surfaces are oriented produce an articulation interface.

The FJR component of the present invention can be any prosthetic capable of at least partially replacing a natural function of a natural facet joint. As noted above, the facet joints are diarthroidal joints that provide both sliding articulation and load transmission features. The facet's articular surfaces contact in extension, limiting rotation and increasing compressive load.

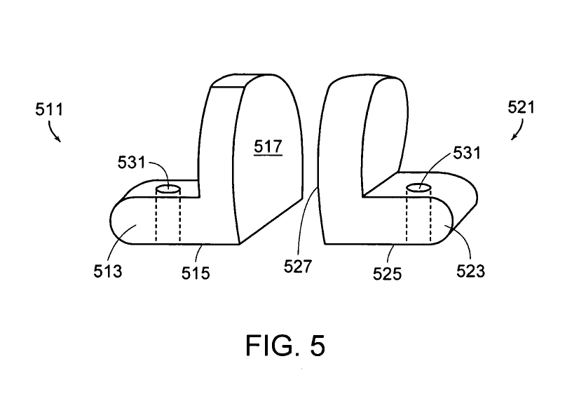

Now referring to FIG. 5, in some embodiments of the present invention, the superior 511 and inferior 521 facet joint components of the prosthesis are independent bodies. In preferred embodiments thereof, the superior facet joint component 511 forms a fixation portion 513 having an outer surface 515 adapted to attach to a first facet and an inner articulation surface 517, while the inferior facet joint component 521 forms a fixation portion 523 having an outer surface 525 adapted to attach to an inferior facet and an inner articulation surface 527. In this embodiment, the inner articulation surfaces are adapted to form an articulation interface. For the purposes of the present invention, this embodiment is called an “articulation prosthesis”. Throughholes 531 are provided in each fixation portion for facilitating the reception of a bone screw to provide bony fixation.

In some articulation embodiments, the first inner articulation surface is convex shaped, while the second inner articulation surface is concave shaped. This creates a ball and socket joint well known in the art.

In some articulation embodiments, each of the first inner articulation surface and second inner articulation surface is cylinder-shaped.

In some articulation embodiments, each of the first inner articulation surface and second inner articulation surface is plane-shaped.

In some embodiments, the first and second articulation surfaces are conforming. In others, the first and second articulation surfaces are non-conforming.

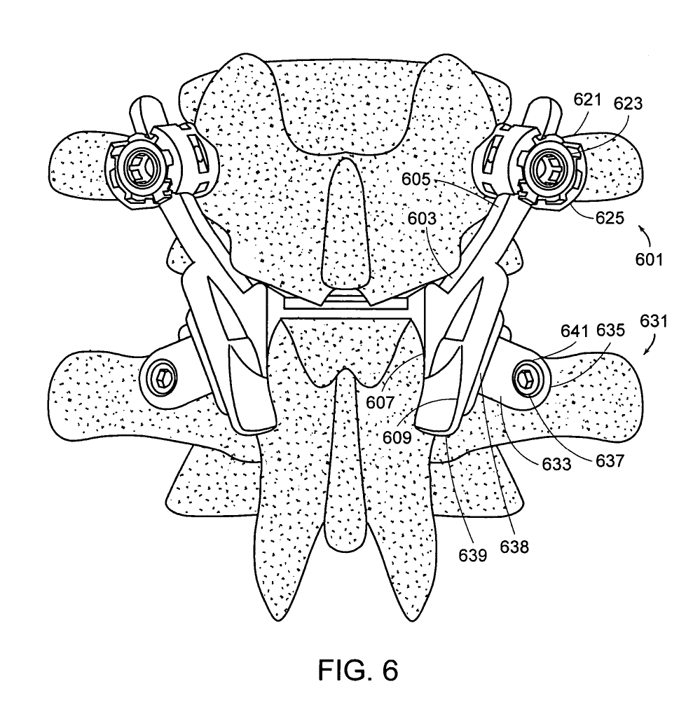

Now referring to FIG. 6, in preferred embodiments, the facet joint replacement component comprises:
a) a superior facet joint component 601 comprising:
   i) a longitudinal body 603 having a superior end portion 605 and an inferior end portion 607, the inferior end portion forming an inner surface 609, and
   ii) a fastener 621 having a distal threadform adapted to fasten to bone and a proximal groove 623 adapted to receive the superior end portion of the longitudinal body, and
   iii) a set screw 625 received within the proximal groove of the fastener,
b) an inferior facet joint component 631 comprising:
   i) a body portion 633,
   ii) outer portion 635 adapted to attach to bone and having a threaded throughhole 637,
   iii) an inner portion having an inner surface 639 adapted to articulate with the inner surface 609 of the superior facet joint component, and
   iv) a fastener 641 received with the threaded throughhole 637.

Still referring to FIG. 6, in preferred embodiments, the superior component 601 of the FJR includes a fastener 623 adapted to fasten to the pedicle portion of the superior vertebral body. The pedicle has been selected as the attachment location because of its clinical proven ability to accept pedicle screws under load, its long term viability and surgical familiarity. The superior end portion is designed to extend from facet region to the upper pedicle, and has a diameter designed to fit approximately within the threadform of the fastener, thereby allowing its fixation. The inferior end portion has an inner surface 609 adapted for bearing against the lower FJR component.

Although FIG. 6 shows set screw 625 as locking only the longitudinal body to the fastener, in other embodiments, the set screw is able to lock both a longitudinal body and ligament to the fastener.

Still referring to FIG. 6, the lower component 631 of the FJR includes a fastener 641 adapted to fasten to the pedicle portion of the lower vertebral body. The pedicle has again been selected as the attachment location. The fastener in this case is a pedicle screw.

As noted above, conventional FJR systems do not appear to allow the surgeon to adjust the distance from the pedicle screw component to the articulation surface component. The FJR system of FIG. 6 has special advantage because it allows for intra-operative adjustability of this distance. In some embodiments, the upper component of the FJR of FIG. 6 comprises a pedicle screw having a groove adapted to receive the superior end portion of the longitudinal body. In use, the pedicle screw is first inserted into the bone, and the superior end portion of the longitudinal body (which in this case, is rod-shaped) is laid into the groove. The surgeon can then slide the longitudinal body relative to the groove until the appropriate position of the inner surface 609 is set. The surgeon can then insert set screw 625 into the groove on top of the superior end portion of the longitudinal body in order to lock the position of the inner surface.

Therefore, in accordance with the present invention, there is provided a facet joint replacement component comprising:
a) a body having an elongate first end portion and a second end portion forming an articulation surface,
b) a fastener having a shank having distal threadform thereon adapted to fasten to a bone, and a proximal end having a transverse groove adapted to receive the elongate first end portion of the longitudinal body,
   wherein a portion of the elongate first end portion of the longitudinal body is slidably received in the groove of the fastener.

The FJR system of FIG. 6 also has special advantage because it allows for intra-operative adjustability of the orientation. In this embodiments, the pedicle screw is a polyaxial screw having a substantially groove adapted to receive the superior end portion of the longitudinal body. The polyaxial nature of this screw allows the surgeon to adjust the orientation of the groove component of the screw, thereby allowing for adjustment of the orientation of the articulation surface.

Therefore, in accordance with the present invention, there is provided a facet joint replacement component comprising:
a) a body having an elongate first end portion and a second end portion forming an articulation surface, and
b) a polyaxial screw adapted for adjustable fixation to the longitudinal body.

The FJR system of FIG. 6 also has special advantage because it reduces or eliminates any moment upon the lower pedicle screw by insuring the articulation forces pass substantially through the lower screw. This is achieved by insuring that the articulation surface of the upper component substantially bisects the lower pedicel screw.

Therefore, in accordance with the present invention, there is provided a facet joint replacement component comprises:
a) a first facet joint component comprising:
   i) a longitudinal body having a first end portion and an second end portion, the inferior end portion forming a first inner articulation surface, and
   ii) means for attaching the longitudinal body to bone, and
b) an second facet joint component comprising:
   i) a body portion,
   ii) outer portion adapted to attach to bone and having a throughhole,
   iii) an inner portion having a second inner articulation surface adapted to articulate with the inner articulation surface of the first facet joint component, and
   iv) a fastener received with the threaded throughhole,
      wherein the first and second inner articulation surfaces are adapted to form an articulation interface defining an articulation force vector, and
wherein the articulation force vector passes through the fastener.

The FJR system of FIG. 6 also has special advantage in that both the superior and inferior components may attach to or abut against another bony structure (e.g., a lamina, pedicle, transverse process or spinous process). The second attachment point may reduce or eliminate a moment upon the pedicle screw.

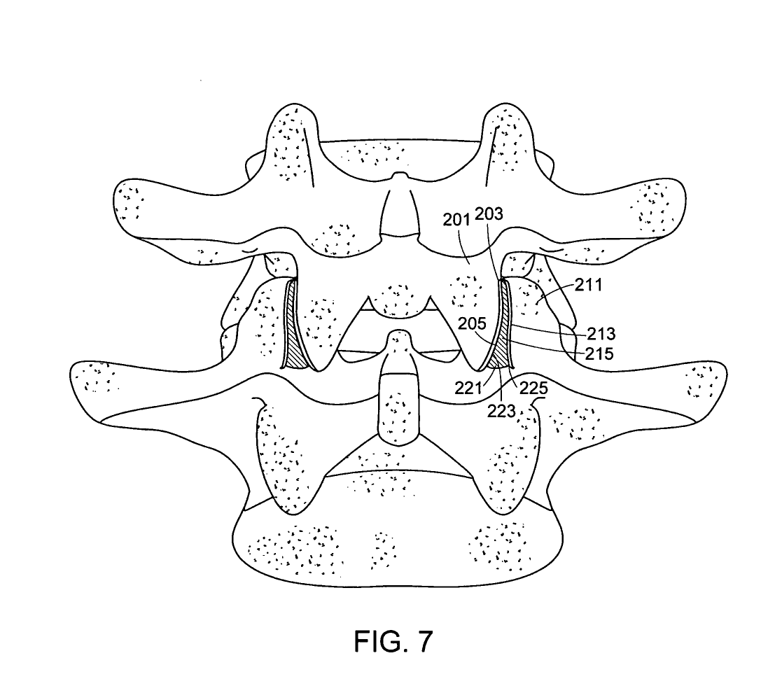

Now referring to FIG. 7, in other embodiments, the superior and inferior facet joint components do not have inner articulation surfaces, but rather are joined by an elastic cushion core. In preferred embodiments thereof, the “cushion-type” prosthesis comprises:
a) a superior facet joint component 201 forming a superior endplate having an outer surface 203 adapted to attach to a superior facet and an inner surface 205,
b) an inferior facet joint component 211 forming an inferior endplate having an outer surface 213 adapted to attach to an inferior facet and an inner surface 215,
c) an elastic core 221 having a superior surface 223 adapted to attach to the inner surface of the superior facet joint component and an inferior surface 225 adapted to attach to the inner surface of the inferior facet joint component.
   For the purposes of the present invention, this embodiment is called a “cushion prosthesis”. In preferred embodiments thereof, the device comprises an elastomer adapted to elastically compress during axial loading and relax when the load is lifted.

In embodiments of the present invention comprising a prosthesis having superior and inferior facet joint components, the fixation portions thereof may comprise an attachment feature. Preferred attachment features are selected from the group consisting of teeth, keels, spikes, pins, holes, and combinations thereof.

In some embodiments, and referring back to FIG. 5, the facet joint replacement comprises:
a) a superior facet joint component 511 having a superior fixation portion 513 having an outer surface 515 adapted to attach to a superior facet, and
b) an inferior facet joint component 521 having an inferior fixation portion 523 having an outer surface 525 adapted to attach to an inferior facet.

In some embodiments, the attachment surfaces of the FJR are adapted to attach to the spinous process. In some embodiments, the attachment surface of the FJR are adapted to attach and/or bear against a lamina. In some embodiments, the attachment surface of the FJR are adapted to attach to a pedicle. In some embodiments, the attachment surface of the FJR are adapted to attach to a transverse process. In some embodiments, the attachment surface of the FJR are adapted to attach to a native facet.

In some embodiments, bony attachment of the attachment surface of the FJR is enhanced by the use of an adhesive, such as fibrin glue or bone cement.

In some embodiments, the fastener and/or the bony attachment surface of an FJR component comprises a material having osteobiologic properties. This material will help the osteointegrative process needed for secure attachment of the fastener and/or the attachment surface to the bone.

In some embodiments, the fastener and/or the attachment surface comprises an orthoconductive portion. The orthoconductive portion typically has a porosity (preferably between about 20 μm and 250 μm) that is adapted to allow the ingress of the osteoconductive cells and an internal surface defined by the porosity that is adapted to attach these cells. In some embodiments, the fastener has an outer surface adapted for bony ingrowth. This outer surface may have an osteoconductive coating thereon, such as a TCP coating or a hydroxyapatite coating.

In some embodiments, the fastener and/or the attachment surface comprises an orthoinductive portion. The orthoinductive portion is preferably a protein, and is more preferably a growth factor. Preferred growth factors include factors from the TGF-beta, IGF-, BMP- and CDMP-families. Preferably, MP52 is selected as the CDMP.

In some embodiments, the fastener and/or attachment surface comprises an orthogenetic portion. The orthogenetic portion preferably comprises mesenchymal stem cells. More preferably, the MSCs are present in a concentration greater than that present in the patient's natural bone marrow.

In some embodiments, the fastener and/or the attachment surfaces may also be coated with other desired agents such as antithrombic or antimicrobial coatings, and pain relievers such as NSAIDS.

In some embodiments, the fastener component of the FJR system is a pedicle screw. In some embodiments, the pedicle screw comprises a longitudinal shank having an integral nut thereon. Distal to the nut, the shank has a first distal threadform thereon and a distal tapered end. Proximal to the integral nut, the shank has a second proximal threadform thereon and a proximal attachment end having a slot.

In some embodiments, the pedicle screw is a polyaxial screw.

In some embodiments, the fastener has a cannulated shank defining a bore that allows for bony ingrowth into the bore. In some embodiments, this bore defines an inner surface adapted for bony ingrowth. This inner surface may have an osteoconductive coating thereon, such as a TCP coating or a hydroxyapatite coating.

The superior and inferior facet joint components of the present invention may be made from any material appropriate for human surgical implantation, including but not limited to all surgically appropriate metals including titanium, titanium alloy, chrome alloys and stainless steel, and non-metallic materials such as carbon fiber materials, resins, plastics and ceramics.

The elastic core, if selected, may comprise polyurethanes, foamed polyethylene, silicones, rubbers, copolymers or hydrogels.

In some embodiments, the FJR component is unilateral. A unilateral FJR at least partially replaces the function of a single facet joint. In some embodiments, the FJR component is bilateral. A bilateral FJR at least partially replaces the function of both facet joints of an FSU.

In some embodiments, the FJR component replaces a single articular process of a facet joint. This replacement process is adapted to articulate with the natural articular process that remains. In some embodiments thereof, the superior process is replaced, while in other the inferior process is replaced.

In other embodiments, the FJR component replaces both articular processes of the facet joint, and these replacement process articulate with each other.

In some embodiments, substantially the entire articular process is replaced with a prosthetic FJR. In others, substantially only the articular surface of the object process is replaced, thereby preserving the underlying bony structure. This replacement surface is adapted to articulate with the natural articular process surface that remains. In some embodiments, the replacement surface comprises a cap.

In some embodiments, the FJR component is a single level FJR. A single level FJR at least partially replaces the function of a single level FSU. In some embodiments, the FJR component is a multi-level FJR. A multi-level FJR at least partially replaces the function of facet joints in at least two levels of FSU.

In some embodiments, the FJR is adapted to replace the natural arch, and so comprises a transverse arch component. In some embodiments, the FJR is adapted to replace a natural process, and so comprises a spinous process component. In some embodiments, the FJR is adapted to replace at least one natural transverse process, and so comprises a transverse process component. In some embodiments, the FJR is adapted to replace at least one pedicle, and so comprises a pedicle component.

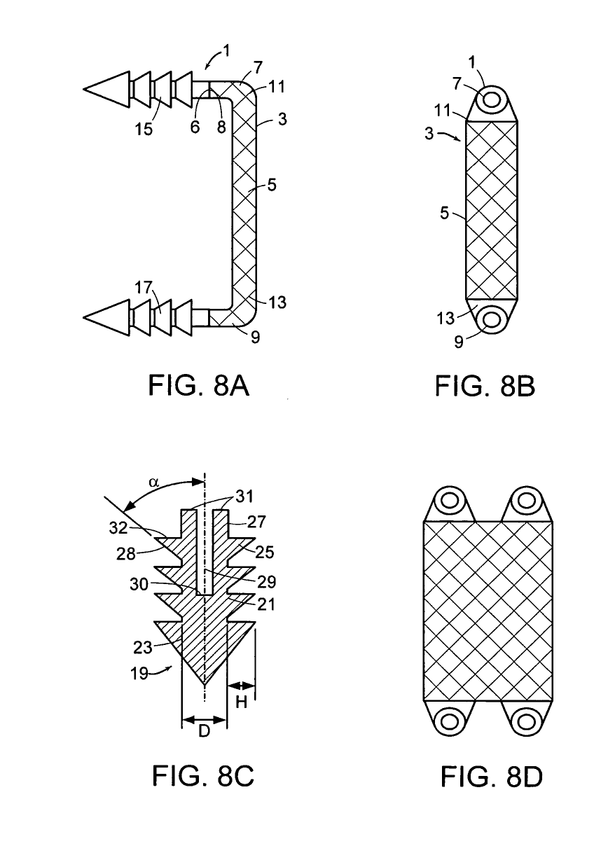

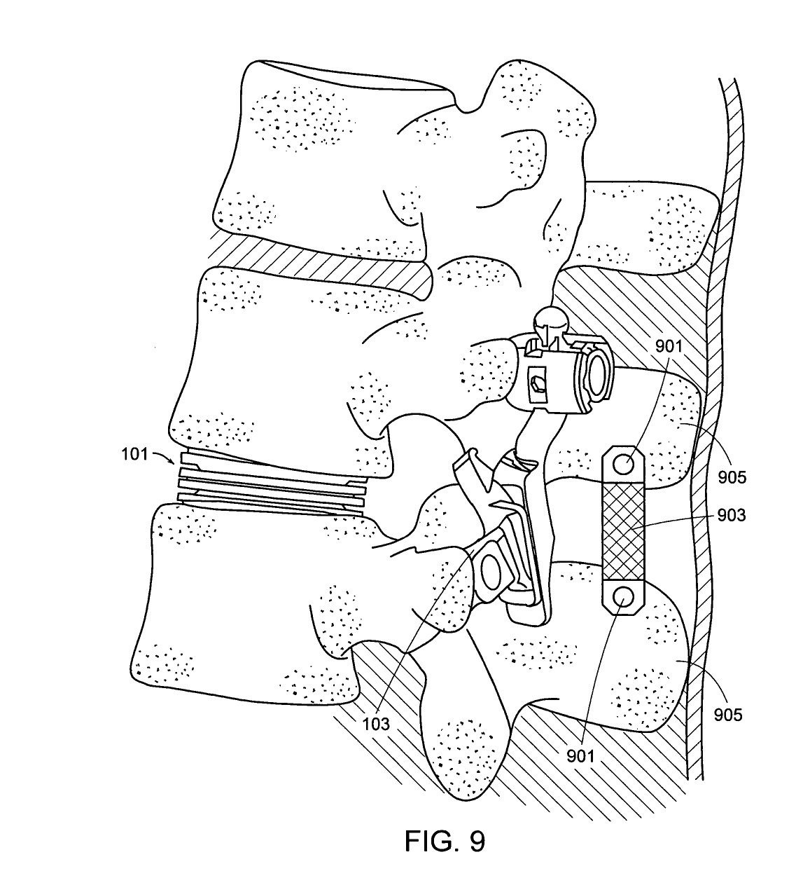

Now referring to FIGS. 8a-8d, there is provided a generic ligament 3 of the present invention:
i) an intermediate portion 5,
ii) first and second end portions 7,9, and
iii) first and second conformable portions 11, 13,
   wherein the first conformable portion is disposed between the intermediate portion and the first end portion, and the second conformable portion is disposed between the intermediate portion and the second end portion, and
iv) first and second fasteners 15,17, and
   wherein the first end portion 7 is shaped to cooperatively connect to the first fastener 15, and the second end portion 9 is shaped to cooperatively connect to the second fastener 17.

In some embodiments, the fasteners are selected from the group consisting of bone screws, hooks, wires, and pins. In some embodiments, the intermediate portion of the ligament is selected from the group consisting of a cable, a wires, an interconnected face, and a soft polymer bonded to the fastener and stretching between the superior and inferior fastener.

In one aspect of the present invention, the facet joint is stabilized in both compression and tension by a prosthetic ligament having fasteners fixated either in the superior and inferior vertebrae or in superior and inferior prosthetic facet joint components. In some embodiments, the fasteners are selected from the group consisting of bone screws, hooks, wires, and pins. In some embodiments, the intermediate portion of the ligament is selected from the group consisting of a cable, a wires, an interconnected face, and a soft polymer bonded to the fastener and stretching between the superior and inferior fastener.

In a preferred embodiment of the present invention, the ligament is shaped as a sheath that can prevent debris produced by the facet articulation from spreading to the surrounding tissues, in particular to various neural structures. Previous facet joint replacement inventions describe resurfacing techniques that replace the contacting faces of the facet joint with metals or polymers. Due to unique variation in motions of the facet joint, these resurfaced contacting faces will inevitably produce wear debris, which is likely to irritate tissues. A membrane or sheath that surrounds the contacting faces and captures generated particles can reduce tissue irritation and inflammation. The membrane or sheath may also have structural integrity in itself and resist over-stretching and thereby supply resistance to tension.

In some embodiments, the width of the sheath is much greater. In preferred sheaths, the sheath is sized to substantially enclose the facet joint. In some embodiments, the sheath is fluid permeable. This feature permits the ingress of fluids that help lubricate the joint, while preventing the egress of wear debris from the facet joint articulation surfaces. In some embodiments, the sheath contains a lubricating fluid, thereby imitating a natural facet joint capsule. In preferred embodiments, the sheath may be pre-assembled prior to implantation, or it may be attached via glues, sutres, wires, thermally activated coagulation or in situ polymer embedding.

In preferred embodiments, this prosthetic facet joint ligament can be attached to anchoring points on opposing sides of a natural or prosthetic facet joint to provide a constraint against relative movement of the facet joints.

The ligament of the present invention can be made of any biocompatible material adapted for constraining but not totally eliminating relative movement between facet joints. In this regard, the facet joint ligament of the present invention mimics the natural facet joint capsule. The ligament of the present invention comprises three features. First it must be adapted to traverse a facet joint. Second, it must allow some flexion to occur across the facet joint. Third, it must resist excessive flexion of the facet joint.

In preferred embodiments, the ligament comprises a pair of attachment end portions and an intermediate portion.

The intermediate portion of the ligament may be adapted to have desirable mechanical qualities found in ligaments, such as elasticity, flexibility, tensionability, and extensibility. Combinations of these qualities allows some displacement of the articular surfaces, but resists excessive displacement.

Preferably, the intermediate portion of the facet joint ligament comprises a nonbioresorbable material including polyesters, (particularly aromatic esters such as polyalkylene terephthalates, polyamides; polyalkenes; poly(vinyl fluoride); polyurethanes; polytetrafluoroethylene (PTFE); carbon fibres; silk; rubber, hydrogels, and glass, and mixtures thereof.

Preferably, the intermediate portion of the facet joint ligament is provided as a fabric. The fabric may be formed by a flat or circular weaving, knitting, braiding, crocheting or embroidery. Preferably, the fabric is braided in order to provide a high tensile strength. Preferred materials suitable for use as fabrics include polyester, polypropylene, polyethylene, carbon fiber, glass, glass fiber, polyurethane, polyaramide, metals, polymers, copolymers, polyactic acid (PLA), polyglycolic acid (PGA), silk, cellusoseic acid, and polycaprolactone fibers.

It is anticipated that, in use, the intermediate portion of the facet joint ligament may rub against soft tissue structures and damage not only those structures but itself as well. Therefore, in some embodiments, the intermediate portion of the facet joint ligament is lubricated. The lubricants lowers the friction coefficient between the ligament and the soft tissue, thereby lowering the wear. Preferred lubricants include hyaluronic acid, proteoglycans, and hydrogels

In some embodiments, the ligament comprises a material having orthobiologic properties. This material will help the body's regenerative processes regrow a natural ligament to replace the prosthetic ligament of the present invention.

In some embodiments, the ligament comprises a material having pain relief properties, such as an NSAID.

In some embodiments, the ligament comprises an orthoconductive portion. The orthoconductive portion typically has a porosity (preferably between about 20 μm and 250 μm) that is adapted to allow the ingress of the osteoconductive cells and an internal surface defined by the porosity that is adapted to attach these cells. In some embodiments, the orthoconductive portion comprises subintestinal submucosa (SIS). In others, it comprises a synthetic polymer.

In some embodiments, the ligament comprises an orthoinductive portion. The orthoinductive portion is preferably a protein, and is more preferably a growth factor. Preferred growth factors include factors from the TGF-beta and IGF-families.

In some embodiments, the ligament comprises an orthogenetic portion. The orthogenetic portion preferably comprises mesenchymal stem cells. More preferably, the MSCs are present in a concentration greater than that present in the patient's natural bone marrow.

In some embodiments, only the intermediate portion of the ligament comprises an orthobiologic material. In some embodiments, only the attachment end portion of the ligament comprises an orthobiologic material. In other embodiments, each of the intermediate and attachment end portions of the ligament comprises an orthobiologic material.

Preferably, the ligament is provided in a sterile form. In some embodiments, the ligment is sterilized, and then placed in a package. Preferably, the inside surface of the package is also sterile.

In some embodiments, the intermediate portion of the ligament is tensionable. A tensionable ligament sags when the ends of the ligaments are moved sufficiently closed to one another so that length of the ligament is less the distance between its ends. This quality allows the opposing facets to move closer to each other under loads without resistance from the ligament. A tensionable ligament also becomes taut when its ends are moved sufficiently away from one another so that length of the ligament is about equal to the distance between its ends. This quality constrains relative movement between the opposing facets. In some embodiments, the tensibility of the ligament is between 5 and 50 N/mm.

In some embodiments, at least a portion of the intermediate portion of the ligament is extensible. An extensible ligament has a first at-rest length when its ends are not loaded, and a second larger length when the ligament is subjected to tensioning. This quality allows the ligament to “give” a predetermined amount under tension. This quality is advantageous because the natural facet joint ligament is also extensible. Preferably, the ligament has an extensibility of between 10% and 30% of the at-rest length of the ligament when subjected to a load of about 250 N. In some embodiments, the extensibility of the ligament is between 5 and 50 N/mm. In other embodiments, the ligament is not extensible.

In some embodiments, at least a portion of the intermediate portion of the ligament is flexible. A flexible ligament bows under axial loading/easily bends under physiologic flexural loading and easily regains its shape when the loading is ceased. This quality allows the ligament to “give’ a predetermined amount while transferring stress under axial loading. This quality is advantageous because the natural facet joint ligament is also flexible. Preferably, the flexible portion of the ligament comprises a curved portion.

Preferably, the ligament is adapted to allow restricted motion of the FSU throughout the life of the patient. However, in the period immediately after the components have been implanted, the human tissue in the wound region has undergone considerable damage and so requires a relatively stable environment in order to heal. In addition, during this early post-operative time period, both the motion disc and the FJR components need to integrate with the bony surfaces to which they are attached, and so also appear to require a relatively stable environment.

Therefore, in some embodiments, the ligament is designed to have time-variable properties. In particular, the ligament is adapted to provide a stiffness that decreases over time. In this condition, the ligament can provide a desirably high stiffness in the immediate post-operative period, thereby stabilizing the region and promoting tissue repair and osteointegration. Once the wounds have healed the components have become integrated, the ligament stiffness decreases, thereby allowing for a desirable range of motion of the FSU.

Preferably, the ligament has a final (6-month) stiffness such that the stiffness of the FSU at that time is in the range of about 1-2 Nm/degree of flexion. Similarly, the ligament preferably has a final (6-month) stiffness such that the stiffness of the FSU at that time is in the range of about 2-3 Nm/degree of extension. In some embodiments, the ligament has an initial stiffness that is between about 2-4 times its final (i.e., 6-month) stiffness. Without wishing to be tied to a theory, it is believed that if the initial stiffness were over about 4 times the final stiffness, fusion would result.

These values provide both the desired high stiffness required for initial stabilization of the region, and long-term flexibility for the FSU.

In some embodiments, the variability in the stiffness of the ligament is accomplished by providing a ligament that experiences significant creep over time. In preferred embodiments, the creeping ligament comprises a polymer, preferably selected from the group consisting of a polyester, a polyolefin and PTFE.

In preferred embodiments, the variability in the stiffness of the ligament is accomplished by providing a resorbable material in the ligament. In some embodiments, the ligament comprises both non-resorbable and resorbable materials. After implantation, each of the non-resorbable and resorbable materials conribute to the initial high stiffness of the ligament. Over time, the resorbable materials degrades away, thereby lowering the ligament stiffness. In some embodiments, the intermediate portion of the ligament comprises non-resorbable fibers and resorbable fibers. In particularly preferred embodiments, the intermediate portion of the ligament comprises a non-resorbable fiber and a resorbable fiber selected from the group consisting PLA, PGA, PLGA, and mixtures thereof.

Each attachment end portion of the ligament is adapted to attach to an anchoring surface on opposite sides of the facet joint. Typically, the attachment end portion comprises a fastener. In other cases, attachment may be provided by sutres or biologically compatible glues. However, in other embodiments, an attachment end portion can simply be terminus being identical in design to the intermediate portion. In such a case, the terminus is inserted into a port located on the anchoring surface, such as a port on a prosthetic having a facet joint articulating surface.

As noted above, in some embodiments, the attachment end portions of the facet joint ligament comprise a pair of fasteners. The function of the fastener is to join to attachment surfaces located on either side of the facet joint in order to securely fasten the intermediate portion of the facet joint ligament across the facet joint. The fastener may be adapted to fasten the facet joint ligament to attachment surfaces located upon either:
a) a facet joint prosthetic component, or
b) a bony surface located adjacent the facet joint prosthetic component.

The attachment end portions of the prosthetic ligament of the present invention may be attached to any two anchoring surfaces on opposite sides of the facet joint, provided the ligament traverses the facet joint. These anchoring surfaces may be located on a bony surface of the opposing vertebrae, or on other prosthetic facet joint components.

In one embodiment, the first attachment end portion of the ligament is adapted to attach to a first load-bearing portion of a facet joint prosthesis. This embodiment is surgeon friendly in that the attachment can be made by the manufacturer prior to surgery, thereby providing ease of use and repeatability.

In some embodiments in which the ligament is attached to a pedicle screw, at least one end of the ligament includes a loop having a diameter slightly larger than the shaft diameter of a pedicle screw. In use, a pilot hole is drilled into the pedicle, the loop is placed over the pilot hole, and the pedicle screw is inserted into the pilot hole, thereby securing the loop therebetween.

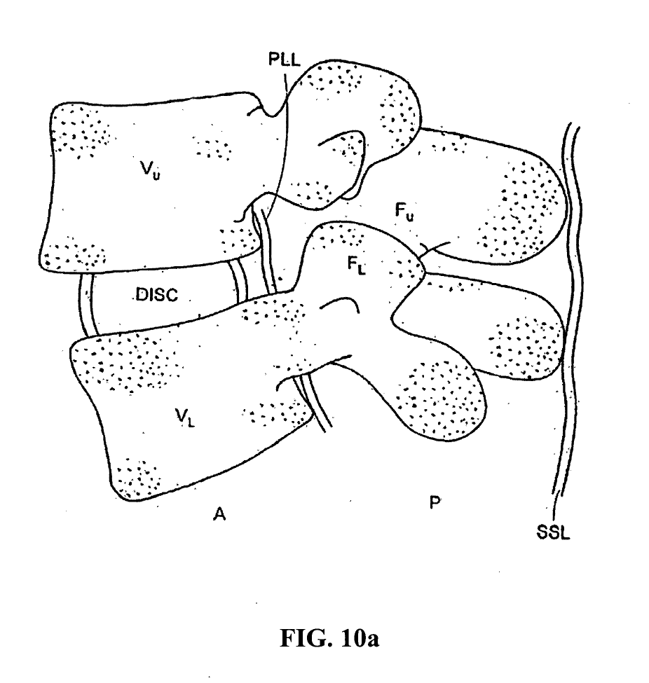

In another embodiment, first attachment end portion of the ligament is adapted to attach to a portion of the natural vertebra. In some embodiments thereof, the vertebral body is used as the anchoring surface. In preferred embodiments thereof, the pedicle portion of the vertebral body is used as the anchoring surface. In another, the facet portion of the vertebra is the anchoring surface. In others, as shown in FIG. 9, a side wall of a spinous process is used as an anchoring surface. In this particular case, each of the upper and lower fasteners 901 of the ligament 903 are respectively attached to the respective side walls 905 of upper and lower spinous processes. In another embodiment, the first end portion of the ligament is adapted to attach to the transverse process. In another embodiment, the first end portion of the ligament is adapted to attach to the pedicle. In another embodiment, the ligament is wrapped around the facet joint.

In embodiments in which at least one attachment end portion of the ligament is attached to bone, the methods described in U.S. Ser. No. 09/822,126, filed Mar. 30, 2001, the specification of which is incorporated by reference in its entirety, may be used.

The fastener may be any design known in the art, including winged, barbed or screw-in mechanisms. Preferably, the fastener is a barbed anchor, as it prevents pullout and is easily inserted. When the attachment surface is a bony surface, the fastener may be a bone fastener.

Now referring to FIG. 8c, preferably, the fastener 19 comprises a longitudinal shank 21, an insertion end 23 comprising protrusions 25 laterally extending from the shank, and an attachment end 27 having an upper surface 31 for connecting to the ligament. Preferably, the lateral protrusions have leading edges 28 which define an angle α of no more than 45 degrees relative to the axis of the shank. In such embodiments, the bearing of the leading edge against the vertebral body surface will not substantially impede the progress of the bone fastener into the bone. Preferably, the leading edges define an angle of no more than 30 degrees, and more preferably between about 20 degrees and 30 degrees. When the angle α is between 20 and 30 degrees, the angle is sufficiently small to not impede the progress of the bone fastener, and yet sufficiently large to insure its secure fit. In some embodiments, the height H of the protrusions on the bone fastener is no more than 70% of the diameter D of the longitudinal shank. When this condition is selected, the risk that any protrusion will act as a shoulder and stop further entry of the bone fastener into the vertebra is minimized. Preferably, the H/D ratio is no more than 40%, more preferably between about 20% and 40%. Within this more preferred window, the protrusion height is sufficiently small to not impede the progress of the bone fastener, and yet sufficiently large to insure its secure fit.

The outer diameter (2H+D) of the bone fastener is preferably between about 3-9 mm, more preferably about 4-6 mm. The length LBF of the bone fastener is preferably between about 3-45 mm, more preferably between about 15-25 mm.

In some embodiments, the attachment end of the bone fastener is made of a ceramic material. When the bone fastener is ceramic, it can withstand the high impact of the driver without failing.

In some embodiments, at least the end portions of the intermediate portion and the attachment end of the bone fastener are made of the same material. When the materials are so selected, these portions may be easily made and pre-connected in an integral fashion. This feature also eliminates the need for sutures.

Referring still to FIG. 8c, in another aspect of the present invention, the attachment end 27 of the fastener is configured to accept a driver element. When this configuration is selected, the bone fastener may be driven into the bone by simply providing axial force to the upper surface 31 of the fastener through the driver. Therefore, in accordance with the present invention, there is provided a facet joint ligament comprising:
a) a ligament comprising first and second end portions, and
b) first and second fasteners,
   wherein the first bone fastener is connected to the first end portion of the ligament, and the second bone fastener is connected to the second end portion of the ligament, and
   wherein the first bone fastener is configured to accept a driver.

Preferably, the configuration defines a recess 29 upon the upper surface 31 of the attachment end 27 of the fastener. This recess 29 is configured to accept the driver (not shown).

In some embodiments, the recess 29 of the bone fastener may be configured to allow insertion of a rescue screw, thereby allowing retrieval of the bone fastener.

In some embodiments, the fastener comprises a material having orthobiologic properties. This material will help the osteointegrative process neeed for secure attachment of the fastener to the bone.

In some embodiments, the fastener surface comprises an orthoconductive portion. The orthoconductive portion typically has a porosity (preferably between about 20 μm and 250 μm) that is adapted to allow the ingress of the osteoconductive cells and an internal surface defined by the porosity that is adapted to attach these cells.

In some embodiments, the fastener comprises an orthoinductive portion. The orthoinductive portion is preferably a protein, and is more preferably a growth factor. Preferred growth factors include factors from the TGF-beta, IGF-, BMP- and CDMP-families. Preferably, MP52 is selected as the CDMP.

In some embodiments, the fastener comprises an orthogenetic portion. The orthogenetic portion preferably comprises mesenchymal stem cells. More preferably, the MSCs are present in a concentration greater than that present in the patient's natural bone marrow.

Preferably, the ligament and fastener components are pre-connected. That is, the components are physically attached prior to their placement upon the spine. Pre-connected components are advantageous because their secured attachment to each other are guaranteed, and the surgeon need not waste time in making the attachment. Components may be pre-connected by physical locking, physical connection, or bonding, or by making the components from the same material and integrally connecting them. When the preconnected components are integrally formed (by, for example, molding or thermoforming), there is no danger of micromotion. Therefore, in accordance with the present invention, there is provided a facet joint ligament comprising:
a) a ligament comprising first and second end portions, and
b) first and second fasteners,
   wherein the first fastener is pre-connected to the first end portion of the ligament, and the second fastener is pre-connected to the second end portion of the ligament.

In some embodiments, at least a portion of the ligament comprises a spring. The spring quality allows the ligament to initially yield to and eventually resist an axial compressive or tension load. In some embodiments, the spring is an expansion spring. In other embodiments, the spring is a compression spring.

In some embodiments of the present invention having both a pair of prosthetic facet joint articulating surfaces and a prosthetic facet joint ligament, the invention limits the natural spinal extension. In these embodiments, extension is limited to no more than 7 degrees, preferably no more than 5 degrees. Preferably, the device produces a spine stiffness is at least 2 Nm/degrees in order to so limit the extension.

In some embodiments of the present invention having a prosthetic facet joint ligament, the invention resists flexion. In these embodiments, flexion is limited to no more than 15 degrees, and preferably is no more than 12 degrees. Preferably, the tensile strength of the prosthetic capsule is between 50 and 300 N, is preferably at least 100 N, and is more preferably at least 200 N.

In some embodiments of the present invention having both a pair of prosthetic facet joint articulating surfaces and a prosthetic facet joint ligament, the invention resists axial rotation. In these embodiments, a pair of devices of the present invention are preferably used so that each facet joint of a functional spine unit has a device, whereby a first device limits the axial rotation while the ligament of the second device is put in tension. This motion tends to produce coupled motion with flexion and lateral bending. In some embodiments, the prosthetic articulating surfaces of the first device are sufficiently strong to withstand compressive forces of at least 100N, and more preferably at least 150N, and more preferably at least 200N.

In some embodiments of the present invention having both a pair of prosthetic facet joint articulating surfaces and a prosthetic facet joint ligament, the invention resists at least anterior-posterior shear. In these embodiments, the prosthetic articulating surfaces contact and the prosthetic articulating surfaces are sufficiently strong to withstand anterior-posterior contact shear forces of at least 500N, and more preferably at least 750N, and more preferably at least 1000N.

In some surgical procedures, such as a laminectomy, the patient's superspinous ligament is often damaged. The SSL is important to the stability of an FSU due to its significant role in restraining patient flexion. Because the SSL possesses the greatest moment about the axis of rotation of any of the spine-related ligaments, damage to the SSL can result in significant instability to the FSU. Therefore, in some embodiments, the ligament of the present invention is adapted to at least partially replace the function of the SSL. In such embodiments, the ligament of the present invention has high flexibility and a high ultimate tensile strength. Preferably, the prosthetic SSL has a tensile strength of at least 50 N, preferably at least 100 N, more preferably at least 150 N, most preferably at least 200 N.

In another embodiment, the ligament of the present invention is adapted to at least partially replace the function of the interspinous ligament (ISL).

In another embodiment, the ligament of the present invention is adapted to at least partially replace the function of the facet joint capsule (FC).

In another embodiment, the ligament of the present invention is adapted to at least partially replace the function of the ligamentum flavum (LF). This embodiment may be selected when the posterior arch is removed.

In another embodiment, the present invention comprises two ligaments adapted to at least partially replace the functions of at least two ligaments selected from the group consisting of the superspinous ligament (SSL), the interspinous ligament (ISL), the facet joint capsule (FC), and the ligamentum flavum.

In another embodiment, the present invention comprises three ligaments adapted to at least partially replace the functions of at least three ligaments selected from the group consisting of the superspinous ligament (SSL), the interspinous ligament (ISL), the facet joint capsule (FC), and the ligamentum flavum.

In another embodiment, the present invention comprises three ligaments adapted to at least partially replace the functions of each of the superspinous ligament (SSL), the interspinous ligament (ISL), the facet joint capsule (FC), and the ligamentum flavum.

The present invention may be used in therapeutic procedures designed to alleviate facet arthritis, stenosis, spondylolysthesis, post-laminectomy kyphosis, and scoliosis.

The present invention may also be used in conjunction with the following surgical procedures: decompressive laminectomy, facet resection, lamina resection, and vertebroplasty.

### EXAMPLE I

This prophetic example will demonstrate one method of implanting the components of the present invention:

In some embodiments, the motion disc is implanted in substantial accordance with the methods described in U.S. Provisional Application US Ser. No. 60/459,280, Hawkins et al., filed Mar. 31, 2004, entitled “Method and Apparatus for Disc Insertion”, Attorney Docket No. 3518.100-001, the specification of which is incorporated by reference in its entirety

First, the surgeon uses a standard posterior approach (either bilateral or unilateral) to uncover the facet joint. Next, the surgeon resects (excises) the facet processes, using standard resection instruments, such as a rongeur or a curette.

Next, the surgeon prepares the surface of each pedicle for insertion of a pedicle screw. This entails locating the appropriate trajectory, probing the pilot hole, and preparing the pedicle surface to receiving the screw.

Next, the surgeon implants the superior pedicle screw into the superior pedicle, and places a looped end of a ligament around the screw head.

Next, the surgeon places the longitudinal portion of the superior component into the groove of the screw head, and then places a set screw on top of that longitudinal portion, effectively securing the longitudinal portion.

The length of the superior component should be such that one surface abuts the natural superior arch. If the surgeon decides to adjust the length of the superior component, the surgeon need only untighten the superior screw and readjust the length as desired.

Now that the location of the superior component has been fixed, the superior component can be used as a template for fixing the location of the inferior component. In particular, the articulation surfaces of the two components are aligned in the desired positions (typically producing a gap therebetween). The alignment should be such that the inferior articulating force travels through the axis of the lower pedicle screw. Once alignment is fixed, the location of the screw hole is marked, and a pilot hole is drilled.

Next, a looped end of a ligament is placed around the pilot hole and the inferior body is oriented to align the pilot hole with the hole in the inferior component.

Lastly, a pedicle screw is inserted through the inferior component and into the pilot hole, thereby fixing the location of the inferior component.

Although some of the facet joint replacement constructs identified above provide the necessary limitations on flexion, extension and rotation of the functional spinal unit, the large size of these constructs may also require that they be implanted through relatively invasive open procedures. In addition, these constructs tend to require complete removal of the facet joint capsule.

Accordingly, in some embodiments of the present invention, the facet joint replacement constructed is designed to allow for its implantation via a surgical technique causing minimal trauma to the facet joint capsule and surrounding soft tissues to the extent possible.

Therefore, in some embodiments, these goals are achieved by replacing the natural facet joint with a construct comprising a bone screw having a head adapted for facet-type articulation.

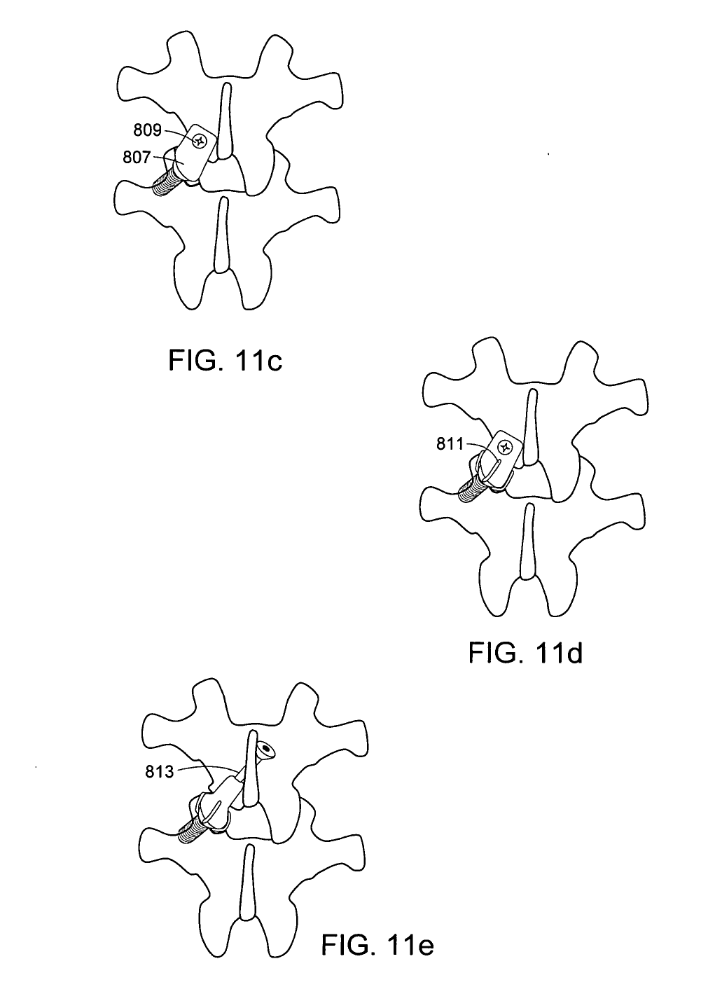

Now referring to FIG. 11a, the surgeon selects a posterior approach with slight lateral-inferior angulation and makes a small incision to access the targeted facet joint. Once access is attained, the surgeon removes the superior facet and the surface of the inferior facet to produce an attachment surface 801 on the remaining portion of the inferior facet.

Now referring to FIG. 11b, the surgeon inserts the inferior facet replacement component into the facet joint by passing the screw through the pedicle and into the vertebral body. In this embodiment, the inferior facet replacement component comprises a bone screw having a threaded shaft 803 and a proximal head 805 adapted for articulation. This approach provides both minimal invasion and a high fixation strength.

Now referring to FIG. 11c, a superior facet replacement 807 is implanted (preferably through the same incision used to implant the inferior component) and is then secured to a surface of the superior lamina. In this case, securement is accomplished by passing a bone screw 809 through the superior component. In preferred embodiments, this superior component comprises a hook (not shown) on its anterior side. In other embodiments, the superior component has a general U-shape to enhance its security to the lamina

Now referring to FIG. 11d, in other minimally invasive embodiments, the MIS superior and inferior components shown above can be connected by at least one ligament 811 to form a pre-assembled facet joint. The addition of a ligament to this system enhances the system's performance by mimicking the action of facet capsular ligaments. In some embodiments, this pre-assembled facet joint is inserted as a whole and fixed as previously described. In other embodiments, this pre-assembled facet joint is inserted piecemeal and assembled in-situ.

Now referring to FIG. 11e, in some embodiments, the superior facet replacement component may be secured to the spine via attachment to a translaminar bolt 813 inserted into the lamina through a second small incision. As above, the anterior side of the superior facet replacement preferably has a hook to grab onto the lamina.

In other minimally invasive embodiments, facet-derived pain is eliminated by denervation and the facet joint replacement component is replaced with a facet joint augmentation component.

In one preferred embodiment thereof, there is provided a method of treating facet joint pathology wherein a primary therapy is first applied to the medial branches and dorsal rami to denervate the nerves in these regions. In some embodiments, the therapy is selected from the group consisting of an energy source (such as pulsed radiofrequency (RF) waves, ultrasound, and microwave), chemical treatment, and freezing.

Upon the completion of the primary therapy, the patient will be positioned to off-load the facet joints and an injectable material (such as a silicone, a polymethsiloxane, a polyurethanes, a hydrogel, and hyaluronic acid) is injected from a syringe into the facet joint to produce a facet joint augmentation within the facet joint. In some embodiments, the injectable material is loaded with at least one therapeutic agent including but not limited: to preservative-free morphine, bupivacaine, tetracaine, opioids, tramadol, ziconotide, betamethasone, clonidine, amitriptyline, fluoxetine, anticonvulsants (such as topiramate), carbamezapine, gabapentin, methlprednisolone acetate morphine3, aminocaproic acid, anti-TNFα molecules,growth factors (such as TGF-b3, TGF-b1, GDF-5) and Cholinesterase inhibitors (such as neostigmine, and glantamine), and combinations thereof. Upon injection, the augmentation of the joint has the effect of distracting the facet joint, re-surfacing the facets and providing a cushion, thereby reducing the pain associated with bone impingement.

Accordingly, in some embodiments, there is provided (claim 32) a method of treating a facet joint, comprising, the steps of:
a) injecting an augmentation material into the facet joint.

In some embodiments, other therapeutic agents such as methlprednisolone acetate morphine3, aminocaproic acid or Anti-TNFa molecules, and/or growth factors such as TGF-b3, TGF-b1, GDF-5 could be injected into the facet joint when the augmentation material is not used.

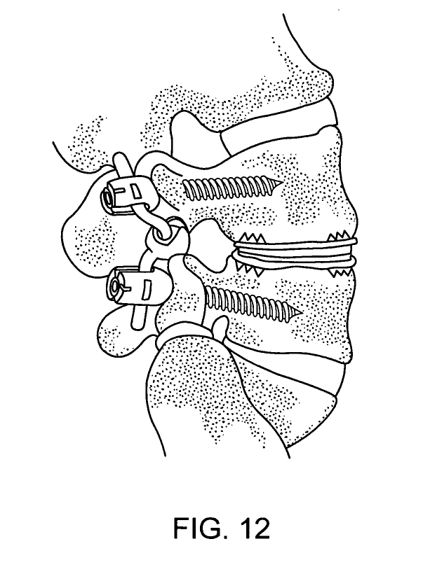

Now referring to FIG. 12, there is provided another embodiment of the present invention in which a portion of the elongate first end portion of the longitudinal body is slidably received in the groove of the fastener (as in FIGS. 1 and 2). In FIG. 12, there is provided a prosthetic facet joint based upon a ball and socket articulation. Pedicle screws are placed superior and inferirorly of the involved level, and hardware connected to the screws as ball and socket joints are positioned in the location of the excised facet joints to restore posterior intervertebral biomechanics.

1. A facet joint replacement component comprising:
a) a body having an elongate first end portion and a second end portion forming an articulation surface,
b) a fastener having a shank having distal threadform thereon adapted to fasten to a bone, and a proximal end having a transverse groove adapted to receive the elongate first end portion of the longitudinal body,
wherein a portion of the elongate first end portion of the longitudinal body is slidably received in the groove of the fastener.

2. The component of claim 1 further comprising:
c) a set screw received within the transverse groove of the fastener.

3. The component of claim 1 wherein the fastener further comprises an integral nut.

4. The component of claim 1 wherein the fastener is a polyaxial screw.

5. The component of claim 1 wherein the fastener is adapted to fasten to a pedicle portion of a vertebral body.

6. The component of claim 1 wherein the elongate first end portion is adapted to extend from a facet region to a pedicle region of a vertebral body.

7. A facet joint replacement system comprising:
a) a superior facet joint component comprising:
i) a longitudinal body having a superior end portion and an inferior end portion, the inferior end portion forming an inner surface, and
ii) a first fastener having a distal threadform adapted to fasten to bone and a proximal groove adapted to receive the superior end portion of the longitudinal body, and
iii) a set screw received within the proximal groove of the fastener,
b) an inferior facet joint component comprising:
i) a body portion,
ii) an outer portion adapted to attach to bone and having a threaded throughhole,
iii) an inner portion having an inner surface adapted to articulate with the inner surface of the superior facet joint component, and
iv) a second fastener received with the threaded throughhole.

8. The system of claim 7 wherein the first fastener further comprises an integral nut.

9. The system of claim 7 wherein the first fastener is a polyaxial screw.

10. The system of claim 7 wherein the first fastener is adapted to fasten to a pedicle portion of a vertebral body.

11. The system of claim 7 wherein the longitudinal body is adapted to extend from a facet region to a pedicle region of a vertebral body.

12. The system of claim 7 wherein the second fastener further comprises an integral nut.

13. The system of claim 7 wherein the second fastener is a polyaxial screw.

14. The system of claim 7 wherein the second fastener is adapted to fasten to a pedicle portion of a vertebral body.

15. The system of claim 7 whererin the inner surface of the inferior facet joint component and the inner surface of the superior facet joint component form a ball and socket.

16. The system of claim 7 wherein the inner surface of the inferior facet joint component and the inner surface of the superior facet joint component each have a cylindrical shape.

17. The system of claim 7 wherein the inner surface of the inferior facet joint component and the inner surface of the superior facet joint component each have a substantially planar shape.

18. A facet joint replacement component kit comprising:
a) a body having an elongate first end portion and a second end portion forming an articulation surface, and
b) a polyaxial screw adapted for adjustable fixation to the longitudinal body.

19. The component of claim 18 further comprising:
c) a set screw received within a transverse groove of the polyaxial screw.

20. The component of claim 18 wherein the screw is adapted to fasten to a pedicle portion of a vertebral body.

21. The component of claim 18 wherein the elongate first end portion is adapted to extend from a facet region to a pedicle region of a vertebral body.

22. A facet joint replacement component comprising:
a) a first facet joint component comprising:
i) a longitudinal body having a first end portion and an second end portion, the second end portion forming a first inner articulation surface, and
ii) means for attaching the longitudinal body to bone, and
b) an second facet joint component comprising:
i) a body portion,
ii) outer portion adapted to attach to bone and having a throughhole,
iii) an inner portion having a second inner articulation surface adapted to articulate with the inner articulation surface of the first facet joint component, and
iv) a fastener received with the threaded throughhole,
wherein the first and second inner articulation surfaces are adapted to form an articulation interface defining an articulation force vector, and
wherein the articulation force vector passes through the fastener.

23. A kit for providing therapy to a functional spinal unit, the unit comprising an upper vertebra having an upper vertebral body and an upper facet, a lower vertebra having a lower vertebral body and a lower facet, the vertebral bodies defining a disc space therebetween, the upper and lower facets defining a facet joint, the kit comprising:
a) a motion disc adapted for insertion into the disc space,
b) a facet joint replacement system adapted for replacement of at least a portion of the facet joint, and
c) a ligament adapted to constrain relative movement between the facets.

24. The kit of claim 23 wherein the motion disc comprises an articulation interface.

25. The kit of claim 23 wherein the motion disc comprises a cushion.

26. The kit of claim 23 wherein the motion disc comprises a spring.

27. The kit of claim 23 wherein the motion disc is adapted for anterior insertion.

28. The kit of claim 23 wherein the motion disc is adapted for posterior insertion.

29. The kit of claim 23 wherein the facet joint replacement comprises an articulation interface.

30. The kit of claim 23 wherein the facet joint replacement system comprises:
i) a superior component adapted to attach to the upper vertebra, and
ii) an inferior component adapted to attach to the lower vertebra.

31. A facet joint replacement system, comprising:
a) an inferior facet replacement component comprising comprisings a bone screw having a threaded shaft and a proximal head adapted for articulation, and
a) a superior facet replacement having an inferior surface adapted for articulation with the proximal head and a superior surface adapted for attachment to a surface of a superior lamina.

32. A method of treating a facet joint, comprising the steps of:
a) injecting an augmentation material into the facet joint.
